(19) United States
(12) Patent Application Publication
(10) Pub. No.: US 2018/0211208 A1
(43) Pub. Date: Jul. 26, 2018

(54) SYSTEMS AND METHODS FOR MONITORING HOME INVENTORY

(71) Applicant: Wal-Mart Stores, Inc., Bentonville, AR (US)

(72) Inventors: David Winkle, Bella Vista, AR (US); Matthew Allen Jones, Bentonville, AR (US); Aaron Vasgaard, Fayetteville, AR (US); Nicholaus Adam Jones, Fayetteville, AR (US); Robert James Taylor, Rogers, AR (US); Todd Davenport Mattingly, Bentonville, AR (US); Bruce W. Wilkinson, Rogers, AR (US); Donald High, Noel, MO (US)

(21) Appl. No.: 15/874,366

(22) Filed: Jan. 18, 2018

Related U.S. Application Data
(60) Provisional application No. 62/448,530, filed on Jan. 20, 2017.

(51) Int. Cl. G06Q 10/08; H04L 12/28; G01N 33/00; G01N 33/02

(52) U.S. Cl. CPC G06Q 10/087; H04L 12/2827; H04L 67/12; G01N 33/02; G01N 33/0004

(57) ABSTRACT

Exemplary embodiments relate to a system for monitoring items in a residence for replenishment. Multiple sensors are disposed in a residence and configured to sense characteristics of an item indicating at least a quality of the item. A computing device is configured to retrieve and analyze usage data for the item. The computing device determines that the item needs replenishment based at least in part on the sensed quality of the item and the usage data for the item. The computing device generates an alert at a user interface in response to determining that the item needs replenishment.

### RELATED APPLICATION

This application claims priority to U.S. Provisional Application No. 62/448,530 filed on Jan. 20, 2017, the content of which is hereby incorporated by reference in its entirety.

### BACKGROUND

Consumers stock up their houses with various items needed for their everyday lifestyle. Some items are used on a daily basis, while others are used less often. Some items are perishable, while others do not expire.

### SUMMARY

In one embodiment, a monitoring system is provided for item replenishment in a residence. The system includes multiple sensors disposed in a residence and configured to sense characteristics of an item. The characteristics indicate at least a quality of the item. The system also includes a computing device equipped with a processor and in communication with the multiple sensors. The computing device is configured to execute a usage module that retrieves and analyzes usage data for the item including at least rate of usage data for the item. The rate of usage data is determined over a period of time using data sensed by the multiple sensors. The computing device is also configured to execute a replenishment module that determines that the item needs replenishment at the residence based at least in part on the sensed quality of the item and the usage data for the item, and generates an alert at a user interface in response to determining that the item needs replenishment.

In one embodiment, a system for monitoring items for replenishment in a residence is provided. The system includes a first array of sensors arranged on a first surface of a shelf holding an item that is configured to sense characteristics of the item indicating at least a quality of the item. The system further includes a second array of sensors arranged on a second surface of the shelf that are configured to sense characteristics of the item on the shelf. The system also includes an interface operatively coupled to the first and second arrays of sensors. The interface is configured to transmit sensor data from the first and second arrays to a computing device. The computing device is equipped with a processor and is configured to execute a usage module that retrieves and analyzes usage data for the item including at least a rate of usage data for the item. The rate of usage data is determined over a period of time using data sensed by the multiple sensors. The computing device is also configured to execute a replenishment module that determines that the item needs replenishment at the residence based at least in part on the sensed quality of the item and the usage data for the item, and generates an alert at a user interface in response to determining that the item needs replenishment.

In another embodiment, a method for monitoring items for replenishment in a residence is provided. The method includes sensing, with multiple sensors disposed in a residence, characteristics of an item in the residence. The characteristics of the item indicate at least a quality of the item. The method further includes retrieving and analyzing, at a usage module, usage data for the item including at least a rate of usage data for the item. The rate of usage data is determined over a period of time using data sensed by the multiple sensors. The method also includes determining, at a replenishment module, that the item needs replenishment at the residence based at least in part on the sensed quality of the item and the usage data for the item, and in response to determining that the item needs replenishment, generating an alert at a user interface.

### BRIEF DESCRIPTION OF DRAWINGS

The accompanying drawings, which are incorporated in and constitute a part of this specification, illustrate one or more embodiments of the invention and, together with the description, help to explain the invention. The embodiments are illustrated by way of example and should not be construed to limit the present disclosure. In the drawings:

FIG. 1 is a block diagram showing a home monitoring system implemented in modules, according to an example embodiment;

FIG. 2 is a flowchart showing an exemplary method for monitoring items in a residence for replenishment, according to an example embodiment;

FIG. 3 is a schematic showing an exemplary home monitoring system at an exemplary storage unit in a residence, according to an example embodiment;

FIG. 4 illustrates a network diagram depicting a system for implementing the home monitoring system, according to an example embodiment; and

FIG. 5 is a block diagram of an exemplary computing device that can be used to implement exemplary embodiments of the home monitoring system described herein.

### DESCRIPTION OF EXEMPLARY EMBODIMENTS

Exemplary embodiments described herein provide a monitoring system for replenishment of items in a residence. An exemplary monitoring system includes multiple sensors disposed in the residence. The sensors sense data indicative of at least quality of the item in the residence. A computing device analyzes usage data for the item including a rate of usage of the item determined over a period of time using data sensed by the multiple sensors. The computing device may determine that the item needs replenishment at the residence based on the sensed quality of the item and the usage data for the item. An alert may be generated at a user interface in response to determining that the item needs replenishment.

In an example embodiment, the home monitoring system described herein includes multiple sensors installed or disposed at various storage areas or units within the residence. For example, multiple sensors may be disposed in a refrigerator, a kitchen cabinet, or a pantry, and the sensors may sense data related to quality of the items stored in these storage areas or units, and may also sense the weight of the items. Moreover, sensors may be disposed at a waste container or recycling container to sense when items or empty item packages are discarded. In this manner, the sensors are disposed at various locations in the residence to enable the home monitoring system to determine when items are consumed or depleted or expired.

Analyzing usage data for items may also enable the home monitoring system to predict when an item may be depleted or consumed, so that the home monitoring system can alert the user to replenish the item before it is completely consumed or depleted. A rate of usage for an item may be determined by analyzing how often an item is purchased for the residence or by analyzing the change in weight or volume of the item. Additionally, a user may provide data that aids in determining a rate of usage for an item. Analyzing quality of an item also enables the home monitoring system to predict when an item may expire, and alerts the user to replenish the item before it expires. When available, the home monitoring system can also determine the expiration date of an item by reading text affixed to the item. A user may also provide his or her preferences to the system on when and how an item should be replenished.

Further, in one embodiment, the home monitoring system may also use data related to temporal events to alert the user to replenish certain items for particular events, such as weather advisories, holiday related events, or special occasions.

In one embodiment, the home monitoring system automatically orders items that need to be replenished at the residence. In one embodiment, the user can input a recipe or list of ingredients, and the home monitoring system determines if an item needs to be replenished based on the recipe.

In some embodiments, the home monitoring system interfaces with one or more in-home voice-assisted speaker systems to access and retrieve user's purchase habits, order history, usage of items, and other data used to determine or predict when an item may need replenishment. The home monitoring system may also interface with one or more in-home voice-assisted speaker systems to automatically order items that need replenishment. Non-limiting examples of in-home voice-assisted speaker systems include various models of Amazon® Echo®, various models of Google® Home®, and other similar systems.

In this manner, the home monitoring system uses sensed data to obtain real-time information on the quality and condition of the items in the residence, in combination with known-data (purchase history, temporal events, etc.) to alert the user when an item needs replenishment. The home monitoring system continually monitors items in the residence, determines when an item needs to be replenished, and alerts the user, preferably before the item is completely consumed, deteriorates or expires, that an item needs to be replenished.

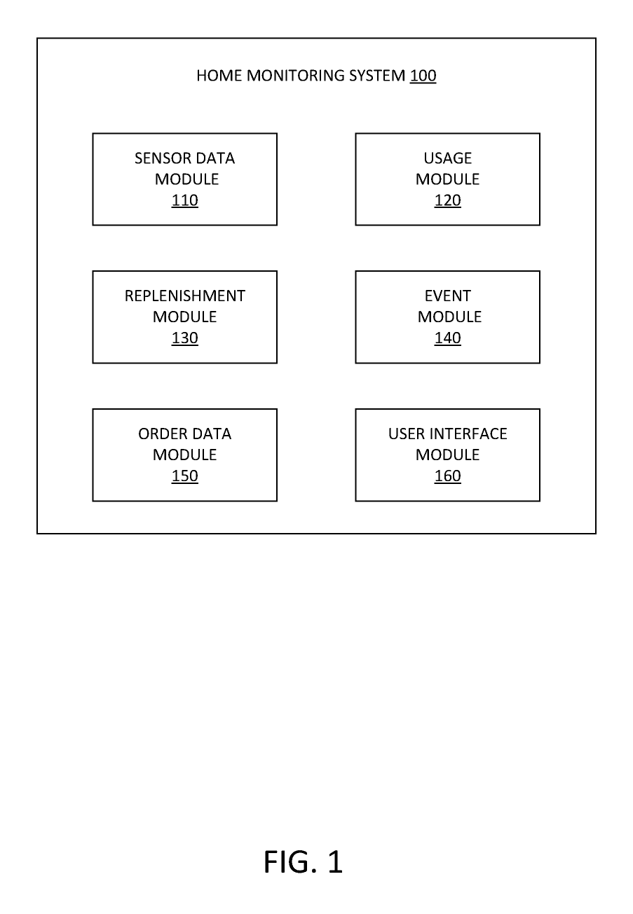

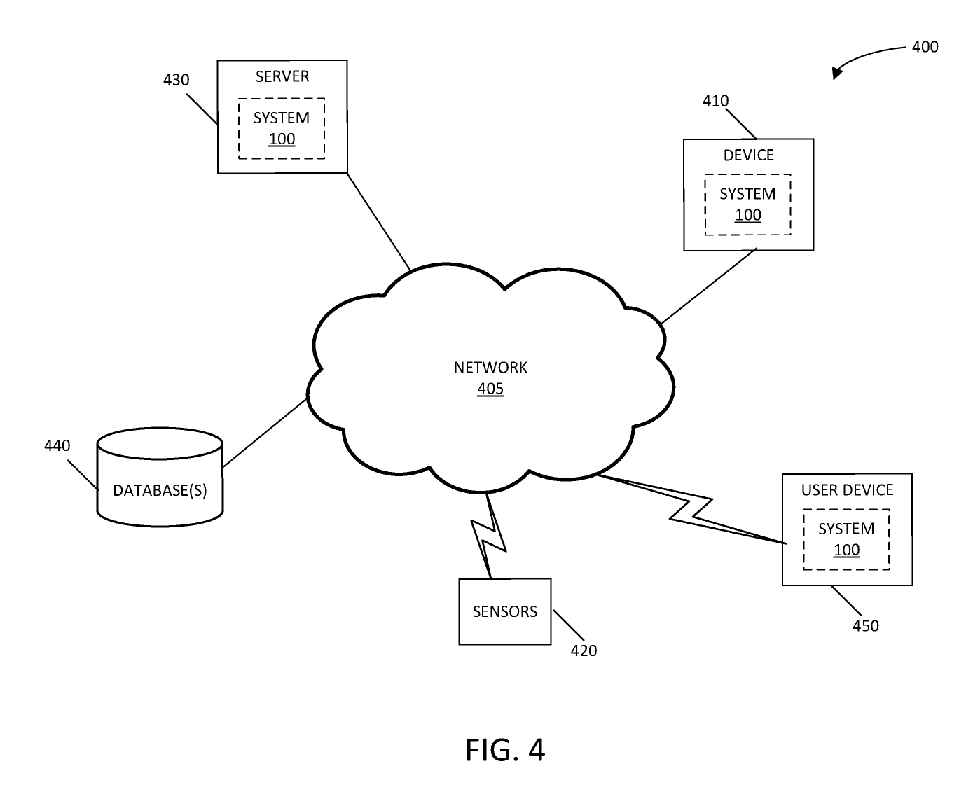

FIG. 1 is a block diagram showing a home monitoring system 100 in terms of functional modules according to an example embodiment. The modules may include a sensor data module 110, a usage module 120, a replenishment module 130, an event module 140, an order data module 150, and user interface module 160. One or more of the modules of system 100 may be implemented in device 410 or server 430 of FIG. 4. The modules may include various circuits, circuitry and one or more software components, programs, applications, or other units of code base or instructions configured to be executed by one or more processors included in device 410 or server 430. Although modules 110, 120, 130, 140, 150, and 160 are shown as distinct modules in FIG. 1, it should be understood that modules 110, 120, 130, 140, 150, and 160 may be implemented as fewer or more modules than illustrated. It should be understood that modules 110, 120, 130, 140, 150, and 160 may communicate with one or more components included in system 400 (FIG. 4), such as device 410, sensors 420, server 430 database(s) 440 or user device 450.

In one embodiment, the sensor data module 110 may be configured to manage and analyze data sensed by the multiple sensors (e.g., sensors 420) disposed in the residence at particular locations. The sensors may be disposed at or near a refrigerator, a kitchen, a kitchen cabinet, a pantry, a waste container, a recycling container, a laundry area, a garage, or other storage areas within the residence. The data sensed by the sensors may indicate freshness of an item, a quality of item, a temperature of item or surroundings, a usage data of item, a weight of item, a gas property indicative of an odor emitted by item, an expiration date of item, a machine-readable data affixed to item, a text affixed to item, a location of item within the residence, a shape of an item, and the like. The sensors disposed in the residence may include a weight sensor, a pressure sensor, a temperature sensor, an off-gassing sensor, a color sensor, a moisture sensor, a location sensor, identifier sensors (e.g., optical label scanner/reader, RFID reader, etc.), image sensing devices, and other sensors. In one embodiment, the sensor data module 120 is included at a computing device (e.g., device 410) or a server (e.g., server 430).

In an example embodiment, one or more sensors are combined together in a container or box that a user can easily place in his residence at desired locations. In another embodiment, the sensors are provided as a “smart shelf” that can be installed in various storage units, for example, in a refrigerator, kitchen cabinet, pantry, and the like. Some embodiments include a sensor matrix including a first array of sensors and a second array of sensors disposed at various locations in the residence.

In one embodiment, the usage module 120 may be configured to analyze sensed data to determine usage data for an item at the residence. The usage module 120 can determine a rate of usage or depletion of an item based on data sensed over a period of time by the multiple sensors disposed in the residence. For example, one or more sensors may be disposed at or near a waste and recycling container to sense items discarded into the containers. The usage module 120 may use this data to determine the rate of usage for the items. As another example, one or more weight sensors may be disposed at storage units within the residence. The usage module 120 may use the change in weight or volume of items to determine a rate of usage for the item. In another embodiment, the usage module 120 may analyze order data to determine usage data for an item.

In one embodiment, the replenishment module 130 may be configured to analyze sensed data to determine that an item requires replenishment. The replenishment module 130 may analyze quality or freshness data sensed by the sensors to determine that an item is expired and requires replenishment. The replenishment module 130 may analyze usage data for an item to determine that an item needs replenishment. The replenishment module 130 may use other factors to determine when an item needs replenishment. The other factors, for example, may include temporal events such as special occasions, weather conditions, holiday related event, sporting event, and the like. The replenishment module 130 may also generate an alert at a user interface in response to determining that an item needs replenishment. The alert indicates to a user a specific item that needs replenishment. In some embodiments, the alert may include further information such as a reason for replenishment (expired item, depleted item, deterioration of quality of an item, an upcoming temporal event, need for a recipe, and the like). In some embodiments, the replenishment module 130 automatically orders the items that need replenishment by programmatically transmitting an order request to a server or a computing device of a retail store. In an example embodiment, the home monitoring system 100 may transmit an order request to an in-home voice-assisted speaker system (such as various models of Amazon® Echo®, Google® Home® or other similar systems) installed or disposed within the residence. The order request may include an amount of item based on the usage data determined by the usage module 120.

In an example embodiment, a user can input a recipe or a list of ingredients and amounts needed for a recipe via a user device (e.g., user device 450). In an example embodiment, a user can input or provide a recipe or a list of ingredients and amounts needed for a recipe via an in-home voice-assisted speaker system (such as various models of Amazon® Echo®, Google® Home® or other similar systems). The home monitoring system 100 can determine if the items on the ingredient list are available in the residence, and if the amount required is available in the residence. If the required amount of an item is not available, then an alert may be generated indicating to the user that a specific item needs replenishment.

In one embodiment, the event module 140 may be configured to manage and analyze temporal events that may occur in the near future that may affect usage of items at the residence. The event module 140 may retrieve information on temporal events, such as weather conditions, sporting events, holiday related events, and the like, from publicly available sources. The event module 140 may retrieve information on special occasions such as birthdays, anniversaries, and the like, from data provided by the user to the home monitoring system 100. Analyzing temporal events enables the home monitoring system 100 to alert the user for item replenishment based on events that may occur in the near future. For example, a birthday may be coming up in a few days, and the home monitoring system 100 can analyze sensed data and determine if certain items (such as ingredients to bake a cake, items to host a party, and the like) need replenishment for the upcoming birthday event. As another example, the event module 140 may identify a weather advisory (such as a tornado, hurricane, snow storm, and the like) for where the residence is located. In this case, the home monitoring system 100 can analyze the sensed data and determine if emergency items (such as candles, non-perishable food items in case power is lost, and the like) need replenishment. In an example embodiment, the home monitoring system 100 stores a list of items in a database that need to be checked for replenishment for each particular event. The list of items may be modified or configured by the user.

In one embodiment, the order data module 150 may be configured to manage and analyze purchase data for the residence. The purchase data may include information on items ordered, quantity of items ordered, time when items were ordered, store from which items were ordered, and the like. The order data module 150 may retrieve or access purchase data for items that are purchased online or purchased in-store. The order data module 150 may have access to purchase data via a user account (e.g., online user account, loyalty or rewards card to use in-store, etc.) setup by one or more users at the residence. Alternatively, the order data module 150 may have access to purchase data via transaction statements for a payment card used by one or more users at the residence to purchase items. In an example embodiment, the order data module 150 may retrieve purchase data via an in-home voice-assisted speaker system (such as various models of Amazon® Echo®, Google® Home® or other similar systems).

In one embodiment, the user interface module 160 may be configured to manage and control displays of multiple user devices to display a user interface for the home monitoring system 100. A user may be able to access the home monitoring system 100 and view alerts via multiple user devices, such as a computing device, a hand-held computing device (tablet), a mobile device, a wearable computing device, and the like. In one embodiment, a user device may be coupled to a storage unit. For example, a user device may be installed on or embedded in a refrigerator door, and the user interface module 160 controls the display of the device on the refrigerator door. As another example, a user device may be installed on or embedded in a cabinet or pantry door and the user interface module 160 controls the display of the device on the cabinet or pantry door.

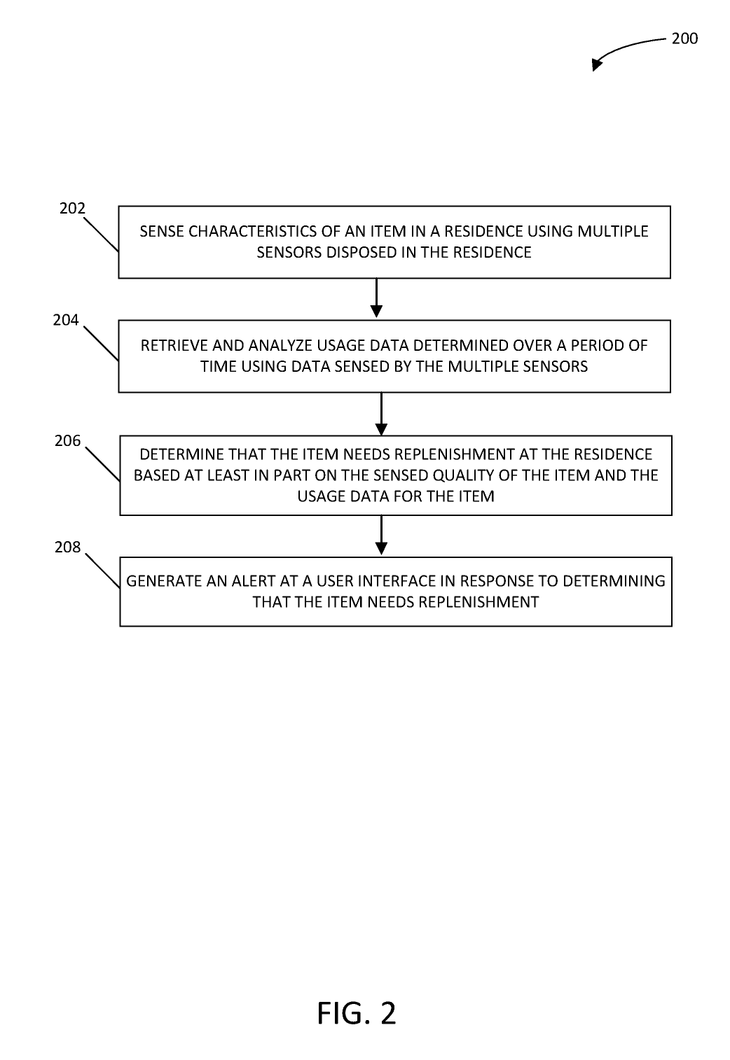

FIG. 2 is a flowchart showing an exemplary method 200 for monitoring items in a residence, according to an example embodiment. The steps of method 200 may be performed by one or more modules shown in FIG. 1. It will be appreciated that the method is programmatically performed by one or more computer-executable processes executing on, or in communication with, one or more computing systems or processors described further below.

At step 202, the sensor data module 110 senses characteristics of an item in a residence using multiple sensors disposed in the residence. The characteristics of the item indicate at least a quality of the item. The characteristics of the item may also indicate a weight of the item, a gas property indicative of an odor of the item, a location of the item, a shape of the item, an expiration date of the item, a unique identifier of the item, and the like. The sensors may be disposed at or near a refrigerator, a kitchen, a kitchen cabinet, a pantry, a waste container, a recycling container, a laundry area, a garage, or other storage areas within the residence. The data sensed by the sensors may indicate freshness of item, a quality of item, a temperature of item or surroundings, a usage data of item, a weight of item, a gas property indicative of an odor emitted by item, an expiration date of item, a machine-readable data affixed to item, a text affixed to item, a location of item within the residence, and the like. The sensors disposed in the residence may include a weight sensor, a thermal imaging sensor, an acoustic sensor a pressure sensor, a temperature sensor, an off-gassing sensor, a color sensor, a moisture sensor, a location sensor, identifier sensors (e.g., optical label scanner/reader, RFID reader, etc.), image sensing devices, and other sensors. In one embodiment the thermal imaging sensor may be a thermal camera enabling temperature to be visually detected. In one embodiment, the sensor data module 110 monitors a differential weight of an item, where increase or decrease in the weight of the item can indicate quality of an item. For example, a perishable item that may have rot may decrease in weight. Similarly, a perishable item that may have grown mold may increase in weight.

At step 204, the usage module 120 retrieves and analyzes usage data for the item. The usage data includes at least a rate of usage for the item that is determined over a period of time using data sensed by the multiple sensors disposed in the residence.

The usage module 120 can determine a rate of usage or depletion of an item based on data sensed over a period of time by the multiple sensors disposed in the residence. For example, one or more sensors may be disposed at a waste and recycling container to sense items discarded into the containers, the rate of usage can be determined using the sensed data. As another example, one or more weight sensors may be disposed at storage units within the residence, and the rate of usage for an item can be determined by sensing the change in weight or volume of the item. The rate of usage data may also be determined by analyzing order data over a period of time to determine how often an item is reordered or repurchased. The usage module 120 may predict or forecast when an item may be depleted.

At step 206, the replenishment module 130 determines whether the item needs replenishment at the residence based at least in part on the sensed quality of the items and the usage data for the item. The replenishment module 130 uses the sensed quality of an item to determine whether the item has expired or has reached/is reaching expiration. The replenishment module 130 uses the usage data for an item to determine when an item is depleted or will be depleted.

The sensed quality of items to determine replenishment needs may be particularly useful for perishable items that have a short-shelf life, such as, produce, meat, seafood, dairy products, bakery items, and the like. The quality of the items may be sensed using an off-gassing sensor, a spectrometer, an odor sensor detecting gas properties indicative of specified odors, a color sensor or the like. The acquired data may be compared against pre-determined parameters for each type of item. The usage data for the item can also be used to determine replenishment needs for perishable items.

The usage data for the item to determine replenishment needs may be particularly useful for non-perishable items such as cleaning supplies, canned food items, and other items with a longer shelf-life. The sensed quality of the items can also be used to determine replenishment needs for non-perishable items.

The sensor data module 110 may also sense or detect an expiration date for the item by scanning a machine-readable code affixed to the item, or by reading the text affixed to the item that indicates the expiration date. The home monitoring system 100 may perform optical character recognition (OCR) on the read text to determine the expiration date for the item. The replenishment module 130 may use the expiration date for the item to determine if an item needs to be replenished. Alternatively, the expiration data may be part of the sensed quality of the items.

At step 208, the replenishment module 140 generates an alert at a user interface in response to determining that the item needs replenishment in step 206. The alert may include an item name. The alert may also include a reason for replenishment, such as, expired item, depleted item, occurrence of a temporal event, and the like.

In one embodiment, the replenishment module 140 is able to determine that an item needs to be replenished before the item is completely consumed, depleted, expended, exhausted or expired, and can alert a user regarding replenishment of the item. Hence, a user may not run out of any items in his residence.

In one embodiment, the replenishment module 140 automatically orders the item for replenishment by programmatically transmitting an order request to a server associated with a store. The quantity of item requested in the order may be based on the usage data of the item, such as rate of usage or historical purchase data.

In one embodiment, multiple sensors are disposed at a laundry area in the residence or an area where laundry is stored. The sensors may sense characteristics of the laundry, such as a gas property indicative of an odor of the laundry, and determine that the clothes need to be washed based on the odor. An alert may be generated at a user interface indicating to the user that the clothes need to be washed. This alert may also be generated based on factors other than odor. For example, the other factors may be weight of the laundry, a weather advisory where loss of power or water is possible, a special occasion that requires certain clothes, an upcoming travel trip that requires clothes, and the like.

In an embodiment, the residence may include two sensor arrays (and possibly three arrays) on various surfaces within the residence for measuring conditions in the storage areas and units in the residence. The first array of sensors may be arranged as multiple individual sensor strips (generally square in shape) extending along a surface of storage unit shelf or wall and defining a sensing grid or matrix. The first array of sensors may also be in the form of lengthy rectangular sensor strips extending along either the x-axis or y-axis of the shelf. In one form, the surface is covered with an appropriate first array of sensors with sufficient discrimination and resolution so that, in combination, the sensors are able to identify the quantity (and optionally the type) of items in the residence.

In one embodiment, the first array of sensors may be formed of piezoelectric material. Piezoelectric sensors are versatile sensors that can measure various characteristics, including pressure, force, and temperature. Also, piezoelectric sensors are fairly sturdy and therefore do not need to be replaced frequently. Although piezoelectric sensors are one suitable sensor type, it should be appreciated that other sensor types may also be used, such as, for example, other types of pressure/weight sensors (load cells, strain gauges, etc.) without departing from the scope of the present invention.

The second array of sensors may be configured to measure at least one of a weight, a pressure, a temperature, and a moisture. The second array of sensors may be arranged on one or more surfaces of the residence, for example a wall in a storage area in the residence. In one embodiment the second array of sensors may be mounted on more than a wall surface so as to generate more data points or sensory data to better monitor conditions of the items. Some or all of the surfaces of the interior of the residence may include sensor arrays so as to define a number of “smart” surfaces.

In an embodiment, the second array of sensors may be arranged in a similar manner along one or more wall surfaces as the first array of sensors is arranged along the shelf surface. For example, the second array of sensors may be arranged as multiple individual sensor strips that collectively define a grid. Further, this second array of sensors may also be formed of piezoelectric material.

The second array of sensors may be temperature sensors that are positioned at different heights along one or more wall surfaces. Under this approach, the first array of sensors may be directed to collecting weight data at the shelf surface, while the second array of sensors is directed to collecting temperature data. The type of sensor in the first and second arrays may be selected so that each array collects a certain type of data that complements the data collected by the other array.

The second array of sensors may include gas emission sensors. These types of sensors are useful in detecting chemicals that may be associated with the deteriorating condition of certain perishable items. Gas emission sensors may also be incorporated into the first array of sensors.

In one embodiment, a third array of sensors may include one or more optical cameras (although other sensor types may also be used). The third array of sensors may include charged-coupled devices, also referred to as CCD camera(s). These digital imaging devices may be selected to be relatively small in size and provide relatively high-quality image data. Alternatively, active-pixel sensors (APS) may also be used (which include CMOS APS sensors). These sensors generally provide lower quality image data but may be less expensive than CCD sensors and use less power. The optical cameras may be positioned at or about the residence to be able to view the items.

In one embodiment, the third array of sensors are configured to identify the type or orientation of item, and this identification may be done in several different ways. For example, regarding type of item, the third array of sensors may be configured to capture images and thereby read barcode labels, recognize text, or recognize color of the item, and/or the third array of sensors may detect RFID tags. In one form, the acquired image data may be compared to item images in an image database to identify the item. As another example, regarding orientation, the third array of sensors may be configured to capture images that show if the item is front facing (as may be desirable), offset with respect to front facing, or may be knocked over and lying on its side.

In one embodiment, the storage areas, such as a refrigerator, cabinets, pantry, etc., in the residence includes shelves to hold the items. The shelf may include a bottom surface and side surfaces, a first array of sensors arranged on the bottom surface of the shelf and configured to measure at least one of weight, pressure, temperature, and moisture, and a second array of sensors arranged on one or more side surfaces of the shelf and configured to measure at least one of weight, pressure, temperature, and moisture. An interface may be operatively coupled to the first and second arrays of sensors, and configured to transmit sensor data from the first and second arrays to a local or remote computing device.

In an embodiment, the first array of sensors may include multiple individual strips defining a grid extending along the bottom surface of the shelf. In some embodiments, the sensors may be built into the shelf itself or may be incorporated into a liner or mat supported by the shelf. Further, the first array of sensors may be formed of piezoelectric material and may be configured to measure weight, pressure, temperature, and moisture at predetermined locations along the bottom surface of the shelf. In addition, the second array of sensors may be disposed at predetermined vertical positions along at least one side surface of the shelf. Also, the second array of sensors may include multiple individual strips defining a grid extending along the at least one side surface of the shelf. Moreover, the first array or second array of sensors may include at least one gas emission sensor.

In one embodiment, the interface may include an RFID device with a memory having a predetermined number of bits equaling the number of sensors in the first array of sensors, and each bit may correspond to a sensor in the first array of sensors. A third array of sensors may be disposed at predetermined vertical positions. In addition, the third array of sensors may be disposed at a top surface of the shelf. Also, the third array of sensors may include one or more optical sensors. The one or more optical sensors may include one or more CCD cameras configured to identify the type or orientation of item based on at least one of barcode labels, RFID tags, text recognition, or color recognition.

The RFID device may have a memory having a predetermined number of bits equaling the number of sensors in the first array of sensors where each bit corresponds to a sensor in the first array of sensors. For example, the first array of sensors may be a 16×16 grid that defines a total of 256 individual sensors, and the first array of sensors may be coupled to a 256 bit RFID device such that each individual sensor corresponds to an individual bit. In addition, the second array of sensors may be arranged in a similar manner. In other words, the sensors may collectively define a 16×16 grid that is coupled to a 256-bit RFID device. It should be appreciated that other array arrangements are possible where there is a 1:1 correspondence between individual sensors and bits of an RFID or memory device.

In one embodiment, the RFID device including a 256 bit memory may be configured to store the location information of the shelf 102 in the residence and location information of items on the shelf 102. Based on detected changes in pressure, weight, and/or temperature, the sensor 109 may configure the corresponding bit of the memory located in the RFID device (as a logic “1” or a logic “0”). The RFID device may then transmit the location of the shelf 102 and data corresponding to changes in the memory to a computing device (e.g., device 410 or server 430).

The computing device may evaluate item freshness levels, particularly for perishable items. When the replenishment module 130 receives sensor data, it may combine it with a sensor reading history and with specific information regarding the item from the database 440. For example, the item information may include data about the item, such as shelf life, to be evaluated in conjunction with sensor readings to allow the calculation of a sell-by date, an estimated expiration date, and/or a stage of ripeness or freshness. In some forms, the replenishment module 130 may determine information regarding the ripeness or freshness left on an item based on predictive comparisons of other similar items. In some embodiments, the computing device may use other known methods to determine the estimated expiration date and/or stage of freshness.

In an embodiment, the sensors are arranged to detect color or texture changes in the items. For example, the arrangement of sensors may include optical sensors (such as in a third array of sensors) that capture image data of the item. It is known that the color and/or texture of certain perishable items, including, for example, certain meat, vegetables and fruit (such as chicken, tomatoes, pears and apples), will change over time. However, non-perishable items may also exhibit color changes over time, due to, for example, fading over time when exposed to sunlight. The optical sensors can take continuous video or still images at certain time intervals, which show the color or texture of the item over time and any changes in color or texture. This image data can be compared to preexisting image data from the database 440 for that particular item that may be associated with preexisting remaining shelf life predictions for the item. Thus, the computing device may be configured to analyze item freshness levels based on the analysis of sensor data over a predetermined amount of time for color or texture changes of the item to determine shelf life.

In another embodiment, the arrangement of sensors may include gas emission sensors (such as in a second array of sensors) that detect gas emissions of chemicals from the item indicating a change in freshness. It is known that certain fruit and other perishable items will emit chemicals indicating ever-decreasing shelf life. The gas emission data can be compared to preexisting data from the database 440 for that particular item that may be associated with preexisting shelf life predictions for that item. The computing device may be configured to analyze item freshness levels based on the analysis of the gas emission data over a predetermined amount of time for chemical changes in the item to determine shelf life.

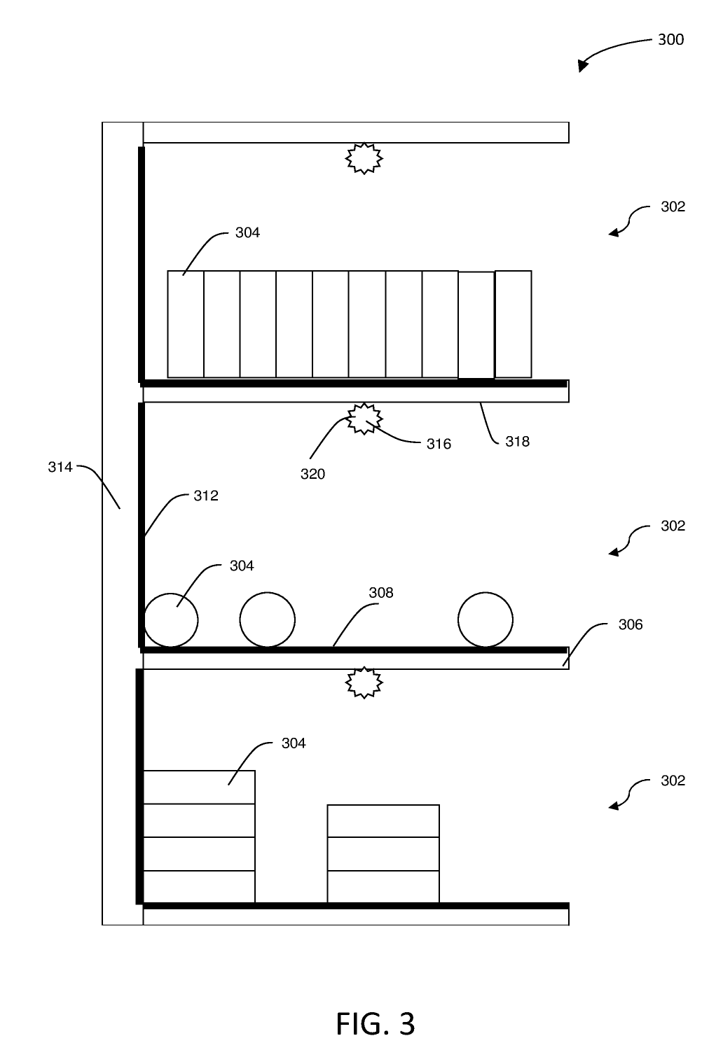

FIG. 3 is a schematic showing an exemplary home monitoring system at an exemplary storage unit 300 in a residence, according to an example embodiment. In FIG. 3, there are shown three shelves 302 supporting various types of items 304. For each shelf 302, the bottom surface 306 includes a first array of sensors 308. In one embodiment, the first array of sensors 308 is selected to measure weight, pressure, temperature, and/or moisture. For example, the first array of sensors 308 may include pressure-sensitive sensors that detect the weight of the items 304 on the shelf 302 being supported by the bottom surface 306.

A second array of sensors 312 is configured to measure at least one of weight, pressure, temperature, and moisture. As shown in FIG. 3, the second array 312 may be arranged on a side (or vertical) surface 314 of the shelf. In this context, it should be understood that the term “side” surface refers to any of the vertical surfaces 314 of the shelf 302, including a front surface, a rear surface, and left and right lateral surfaces. Further, although FIG. 3 shows the second array of sensors 312 disposed on the side (rear) surface 314, it should be understood that the second array of sensors 312 may be disposed on more than one side (or vertical) surface 314.

A third array of sensors 316 may be disposed at certain vertical position(s) on or about the shelf 302. In FIG. 3, the third array of sensors 316 is shown as disposed at a top surface 318 of the shelf 302. More specifically, in this example, the third array of sensors 316 includes one sensor 320 that is mounted directly above the shelf 302. It should be understood that the third array of sensors 316 may include several individual sensors and may be mounted at different heights on or about the shelf 302.

FIG. 4 illustrates a network diagram depicting a system 400 used for implementing the home monitoring system, according to an example embodiment. The system 400 can include a network 405, multiple devices, for example device 410 and a device 450, multiple sensors 420, a server 430, and database(s) 440. Each of components 410, 420, 430, 440 and 450 is in communication with the network 405. It will be appreciated that the depicted components may be combined in combinations other than those illustrated herein without departing from the scope of the present invention. For example, the functionality of the device 410 and server 430 may be combined in a single computing system or device.

In an example embodiment, one or more portions of network 405 may be an ad hoc network, an intranet, an extranet, a virtual private network (VPN), a local area network (LAN), a wireless LAN (WLAN), a wide area network (WAN), a wireless wide area network (WWAN), a metropolitan area network (MAN), a portion of the Internet, a portion of the Public Switched Telephone Network (PSTN), a cellular telephone network, a wireless network, a WiFi network, a WiMax network, any other type of network, or a combination of two or more such networks.

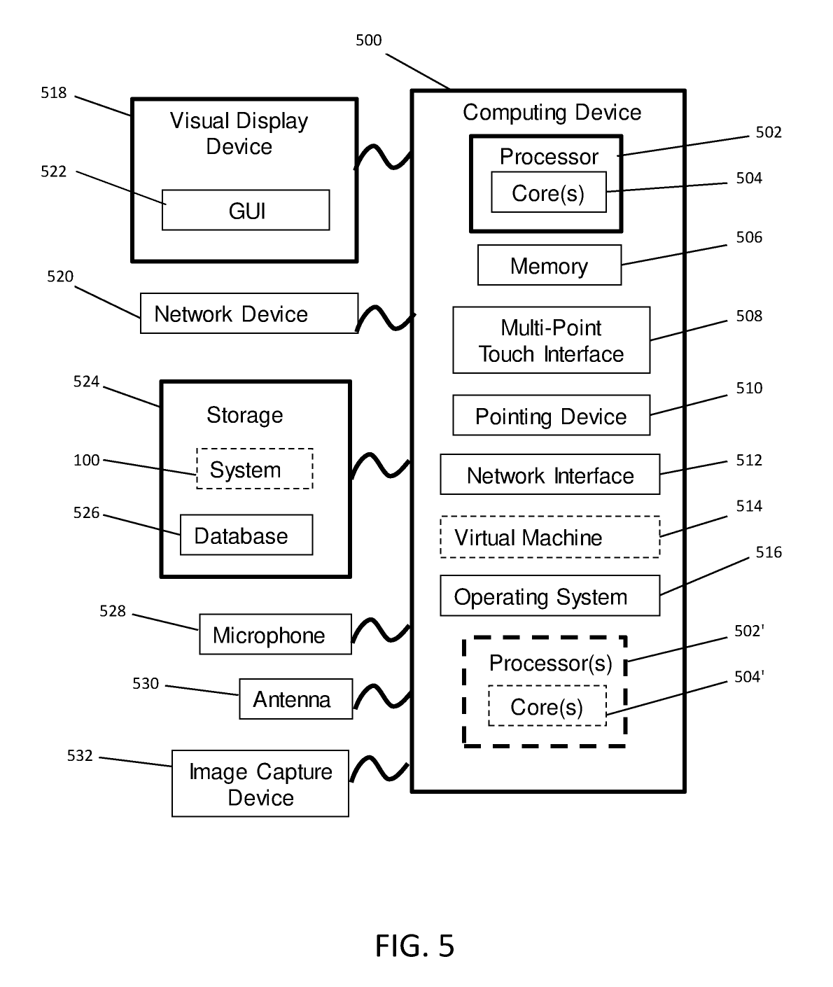

The device 410 may include, but is not limited to, work stations, computers, general purpose computers, Internet appliances, hand-held devices, wireless devices, portable devices, wearable computers, cellular or mobile phones, portable digital assistants (PDAs), smart phones, tablets, ultrabooks, netbooks, laptops, desktops, multi-processor systems, microprocessor-based or programmable consumer electronics, game consoles, set-top boxes, network PCs, mini-computers, and the like. The device 410 can include one or more components described in relation to computing device 500 shown in FIG. 5.

The user device 450 may include, but is not limited to, work stations, computers, general purpose computers, Internet appliances, hand-held devices, wireless devices, portable devices, wearable computers, cellular or mobile phones, portable digital assistants (PDAs), smart phones, tablets, ultrabooks, netbooks, laptops, desktops, multi-processor systems, microprocessor-based or programmable consumer electronics, game consoles, set-top boxes, network PCs, mini-computers, computing device installed in a vehicle, a vehicle navigation and computing system, an embedded computing system, a computing system installed or embedded in residential appliances, a computing system installed or embedded in residential storage units, an in-home voice-assisted speaker system, and the like. The device 450 can include one or more components described in relation to computing device 500 shown in FIG. 5. In an example embodiment, the user device 450 is a device used by a user in the residence.

The device 410, 450 may connect to network 405 via a wired or wireless connection. In an example embodiment, the device 410, 450 include one or more components of the home monitoring system 100 described herein, and may perform one or more functionalities described herein. For example, the device 410 may determine when an item requires replenishment. For example, the device 450 may display an alert generated by the home monitoring system 100 indicating a need for replenishing an item.

In an example embodiment, some of the components of the home monitoring system 100 may be included in the device 410, while the other components are included in the server 430. Some of the functionalities of the home monitoring system described herein may be performed by the server 430.

The sensors 420 disposed in the residence may include a weight sensor, a pressure sensor, a temperature sensor, a thermal imaging sensor, an acoustic sensor, an off-gassing sensor, a color sensor, a moisture sensor, a location sensor, identifier sensors (e.g., optical label scanner/reader, RFID reader, etc.), an image sensing device, an optical sensor, a humidity sensor, and other sensors.

Each of the database(s) 440 and server 430 may be connected to the network 405 via a wired or wireless connection. The server 430 may include one or more computing devices or processors configured to communicate with the device 410, the sensors 420, database(s) 440, and the user device 450 via network 405. The server 430 may host one or more applications or websites accessed by the device 410, 450 and/or facilitate access to the content of database(s) 440. Database(s) 440 include one or more storage devices for storing data and/or instructions (or code) for use by the device 410, 450 and server 430, such as data sensed by the sensors 420, usage data, purchase data, data related to temporal events, item data, and the like. Database(s) 440, and/or server 430, may be located at one or more geographically distributed locations from each other or from the device 410. Alternatively, database(s) 440 may be included within server 430.

FIG. 5 is a block diagram of an exemplary computing device 500 that may be used to implement exemplary embodiments of the home monitoring system 100 described herein. The computing device 500 includes one or more non-transitory computer-readable media for storing one or more computer-executable instructions or software for implementing exemplary embodiments. The non-transitory computer-readable media may include, but are not limited to, one or more types of hardware memory, non-transitory tangible media (for example, one or more magnetic storage disks, one or more optical disks, one or more flash drives), and the like. For example, memory 506 included in the computing device 500 may store computer-readable and computer-executable instructions or software for implementing exemplary embodiments of the home monitoring system 100. The computing device 500 also includes configurable and/or programmable processor 502 and associated core 504, and optionally, one or more additional configurable and/or programmable processor(s) 502′ and associated core(s) 504′ (for example, in the case of computer systems having multiple processors/cores), for executing computer-readable and computer-executable instructions or software stored in the memory 506 and other programs for controlling system hardware. Processor 502 and processor(s) 502′ may each be a single core processor or multiple core (504 and 504′) processor.

Virtualization may be employed in the computing device 500 so that infrastructure and resources in the computing device may be shared dynamically. A virtual machine 514 may be provided to handle a process running on multiple processors so that the process appears to be using only one computing resource rather than multiple computing resources. Multiple virtual machines may also be used with one processor.

Memory 506 may include a computer system memory or random access memory, such as DRAM, SRAM, EDO RAM, and the like. Memory 506 may include other types of memory as well, or combinations thereof.

A user may interact with the computing device 500 through a visual display device 518, such as a computer monitor, which may display one or more graphical user interfaces 522 that may be provided in accordance with exemplary embodiments. The computing device 500 may include other I/O devices for receiving input from a user, for example, a keyboard or any suitable multi-point touch interface 508, a pointing device 510 (e.g., a mouse), a microphone 528, and/or an image capturing device 532 (e.g., a camera or scanner). The multi-point touch interface 508 (e.g., keyboard, pin pad, scanner, touch-screen, etc.) and the pointing device 510 (e.g., mouse, stylus pen, etc.) may be coupled to the visual display device 518. The computing device 500 may include other suitable conventional I/O peripherals.

The computing device 500 may also include one or more storage devices 524, such as a hard-drive, CD-ROM, or other computer readable media, for storing data and computer-readable instructions and/or software that implement exemplary embodiments of the home monitoring system 100 described herein. Exemplary storage device 524 may also store one or more databases for storing any suitable information required to implement exemplary embodiments. For example, exemplary storage device 524 can store one or more databases 526 for storing information, such as data sensed by the sensors 420, order data, item data, pickup and delivery addresses, and/or any other information to be used by embodiments of the system 100. The databases may be updated manually or automatically at any suitable time to add, delete, and/or update one or more items in the databases.

The computing device 500 can include a network interface 512 configured to interface via one or more network devices 520 with one or more networks, for example, Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections, controller area network (CAN), or some combination of any or all of the above. In exemplary embodiments, the computing device 500 can include one or more antennas 530 to facilitate wireless communication (e.g., via the network interface) between the computing device 500 and a network. The network interface 512 may include a built-in network adapter, network interface card, PCMCIA network card, card bus network adapter, wireless network adapter, USB network adapter, modem or any other device suitable for interfacing the computing device 500 to any type of network capable of communication and performing the operations described herein. Moreover, the computing device 500 may be any computer system, such as a workstation, desktop computer, server, laptop, handheld computer, tablet computer (e.g., the iPad™ tablet computer), mobile computing or communication device (e.g., the iPhone™ communication device), internal corporate devices, computing devices installed in a vehicle, or other form of computing or telecommunications device that is capable of communication and that has sufficient processor power and memory capacity to perform the operations described herein.

The computing device 500 may run operating system 516, such as versions of the Microsoft® Windows® operating system, different releases of the Unix and Linux operating systems, versions of the MacOS® for Macintosh computers, embedded operating systems, real-time operating systems, open source operating systems, proprietary operating systems, or other operating systems capable of running on the computing device and performing the operations described herein. In exemplary embodiments, the operating system 516 may be run in native mode or emulated mode. In an exemplary embodiment, the operating system 516 may be run on one or more cloud machine instances.

The following description is presented to enable any person skilled in the art to create and use a computer system configuration and related method and article of manufacture for a home monitoring system. Various modifications to the example embodiments will be readily apparent to those skilled in the art, and the generic principles defined herein may be applied to other embodiments and applications without departing from the spirit and scope of the invention. Moreover, in the following description, numerous details are set forth for the purpose of explanation. However, one of ordinary skill in the art will realize that the invention may be practiced without the use of these specific details. In other instances, well-known structures and processes are shown in block diagram form in order not to obscure the description of the invention with unnecessary detail. Thus, the present disclosure is not intended to be limited to the embodiments shown, but is to be accorded the widest scope consistent with the principles and features disclosed herein.

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes multiple system elements, device components or method steps, those elements, components or steps may be replaced with a single element, component or step. Likewise, a single element, component or step may be replaced with multiple elements, components or steps that serve the same purpose. Moreover, while exemplary embodiments have been shown and described with references to particular embodiments thereof, those of ordinary skill in the art will understand that various substitutions and alterations in form and detail may be made therein without departing from the scope of the invention. Further still, other embodiments, functions and advantages are also within the scope of the invention.

Exemplary flowcharts are provided herein for illustrative purposes and are non-limiting examples of methods. One of ordinary skill in the art will recognize that exemplary methods may include more or fewer steps than those illustrated in the exemplary flowcharts, and that the steps in the exemplary flowcharts may be performed in a different order than the order shown in the illustrative flowcharts.

What is claimed is:

1. A monitoring system for item replenishment in a residence, the system comprising:
a plurality of sensors disposed in a residence and configured to sense characteristics of an item indicating at least a quality of the item;
a computing device equipped with a processor and in communication with the plurality of sensors, the computing device configured to execute:
a usage module configured to retrieve and analyze usage data for the item including at least rate of usage data for the item, the rate of usage data determined over a period of time using data sensed by the plurality of sensors; and
a replenishment module configured to:
determine that the item needs replenishment at the residence based at least in part on the sensed quality of the item and the usage data for the item, and
generate an alert at a user interface in response to determining that the item needs replenishment.

2. The monitoring system of claim 1, wherein the computing device is further configured to execute an event module configured to retrieve and analyze event data related to one or more temporal events, the temporal events including at least one of a weather related event, holiday event, special occasion, and sporting event,
wherein the replenishment module determines that the item needs replenishment based at least in part on the event data.

3. The system of claim 1, wherein the plurality of sensors include at least one of a weight sensor, a pressure sensor, a temperature sensor, thermal imaging sensor, an off-gassing sensor, a color sensor, an acoustic sensor, and a moisture sensor at predetermined locations in the residence.

4. The system of claim 1, wherein the plurality of sensors are disposed at or near at least one of a refrigerator, a kitchen, a kitchen cabinet, a pantry, a waste container, and a laundry area.

5. The system of claim 1, further comprising a sensor data module configured to scan an optical machine readable code affixed to the item using an optical scanner disposed at the residence.

6. The system of claim 1, wherein the alert is displayed on the user interface of a device affixed to a refrigerator in the residence.

7. The system of claim 1, wherein in response to determining that the item needs replenishment, the replenishment module is configured to transmit an order request for a specific quantity of the item to a server of a retail store, and wherein the quantity of the item is determined based on the usage data for the item.

8. The system of claim 1, wherein one or more of the plurality of sensors are disposed at a waste container in the residence, and the usage data for the item is determined using data sensed by the one or more plurality of sensors, the data indicating a quantity of the item that is disposed in the waste container.

9. A system for monitoring items for replenishment in a residence, the system comprising:
a first array of sensors arranged on a first surface of a shelf holding an item and configured to sense characteristics of the item indicating at least a quality of the item;
a second array of sensors arranged on a second surface of the shelf and configured to sense characteristics of the item on the shelf;
an interface operatively coupled to the first and second arrays of sensors, the interface configured to transmit sensor data from the first and second arrays to a computing device; and
wherein the computing device is equipped with a processor and is configured to execute:
a usage module configured to retrieve and analyze usage data for the item including at least rate of usage data for the item, the rate of usage data determined over a period of time using data sensed by the first and second array of sensors, and
a replenishment module configured to:
determine that the item needs replenishment at the residence based at least in part on the sensed quality of the item and the usage data for the item, and
generate an alert at a user interface in response to determining that the item needs replenishment.

10. A method for monitoring items for replenishment in a residence, the method comprising:
sensing, with a plurality of sensors disposed in a residence, characteristics of an item in the residence, the characteristics of the item indicating at least a quality of the item;
retrieving and analyzing, at a usage module, usage data for the item including at least a rate of usage data for the item, the rate of usage data determined over a period of time using data sensed by the plurality of sensors;
determining, at a replenishment module, that the item needs replenishment at the residence based at least in part on the sensed quality of the item and the usage data for the item; and
in response to determining that the item needs replenishment, generating an alert at a user interface.

11. The method of claim 10, further comprising:
retrieving and analyzing, at an event module, data related to one or more temporal events, the temporal events including at least one of a weather related event, holiday event, special occasion, and sporting event; and
determining at the replenishment module that the item needs replenishment based in part on the event data.

12. The method of claim 10, wherein the plurality of sensors include at least one of a weight sensor, a pressure sensor, a temperature sensor, an off-gassing sensor, a color sensor, and a moisture sensor at predetermined locations in the residence.

13. The method of claim 10, wherein the plurality of sensors are disposed at or near at least one of a refrigerator, a kitchen, a kitchen cabinet, a pantry, a waste container, and a laundry area.

14. The method of claim 10, further comprising:
sensing an expiration date for the item using an image capturing device disposed at the residence.

15. The method of claim 14, further comprising:
sensing freshness of the item using one or more of the plurality of sensors in the residence;
determining that the item is approaching an expiration date based on the sensed freshness data and the sensed expiration date for the item; and
generating the alert at the user interface in response to the determining.

16. The method of claim 10, further comprising:
scanning an optical machine readable code affixed to the item using an optical scanner disposed at the residence.

17. The method of claim 10, wherein the alert is displayed on the user interface of a device affixed to a refrigerator in the residence.

18. The method of claim 10, further comprising:
transmitting an order request for a specific quantity of the item to a server of a retail store in response to determining that the item needs replenishment, wherein the specific quantity of the item is determined based on the usage data for the item.

19. The method of claim 10, wherein one or more of the plurality of sensors are disposed at a waste container in the residence, and the quantity of the item is determined using data sensed by the one or more plurality of sensors, the data indicating a quantity of the item that is disposed in the waste container.

20. The method of claim 10, wherein one or more of the plurality of sensors are disposed at a laundry area, and the method further comprises:
sensing a gas property indicative of an odor using the one or more of the plurality of sensors;
determining that the sensed gas property indicative of an odor meets a predefined criteria; and
generating a laundry alert at the user interface in response to the determining.

21. The method of claim 10, further comprising:
receiving a list of items and a listed quantity for each item in the list;
determining an available quantity in the residence for each item in the list; and
generating the alert when the listed quantity is more than the available quantity.

22. The method of claim 10, wherein the usage data of the item further includes at least one order data for the item, purchase data for the item, and user-defined preferences for the item.
